(19) United States
(12) Patent Application Publication
(10) Pub. No.: US 2009/0042265 A1
(43) Pub. Date: Feb. 12, 2009

(54) Thermophilic Microorganisms with Inactivated Lactate Dehydrogenase Gene (LDH) for Ethanol Production

(76) Inventors: Anthony Atkinson, Guildford (GB); Roger Cripps, Guilford (GB); Kirstin Eley, Guildford (GB); Brian Rudd, Welwyn Garden City (GB); Martin Todd, Welwyn Garden City (GB); Ann Thompson, Takapuna (NZ)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/913,480

(22) PCT Filed: May 3, 2006
(86) PCT No.: PCT/GB2006/001586

§ 371 Date: Apr. 17, 2008

(30) Foreign Application Priority Data
May 4, 2005 (GB) 0509068.3
Jun. 7, 2005 (GB) 0511603.3

(51) Int. Cl. C12P 7/06; C12N 1/20; C12N 15/00

(52) U.S. Cl. 435/161; 435/243; 435/252.5; 435/252.31; 435/320.1

(57) ABSTRACT

A mutated thermophilic microorganism is prepared, with a modification to inactivate the lactate dehydrogenase gene of a wild-type microorganism. The mutated microorganism is used in the production of ethanol, utilising C₃, C₅ or C₆ sugars as the substrate.

### FIELD OF THE INVENTION

This invention relates to the production of ethanol as a product of bacterial fermentation. In particular, the invention relates to ethanol production by thermophilic bacteria.

### BACKGROUND TO THE INVENTION

Bacterial metabolism can occur through various different mechanisms depending on the bacterial species and environmental conditions. Hetrotrophic bacteria, which include all pathogens, obtain energy from oxidation of organic compounds, with carbohydrates (particularly glucose), lipids and protein being the most commonly oxidised compounds. Biologic oxidation of these organic compounds by bacteria results in synthesis of ATP as the chemical energy source. The process also permits generation of more simple organic compounds (precursor molecules) which are required by the bacterial cell for biosynthetic reactions. The general process by which bacteria metabolise suitable substrates is glycolysis, which is a sequence of reactions that converts glucose into pyruvate with the generation of ATP. The fate of pyruvate in the generation of metabolic energy varies depending on the microorganism and the environmental conditions. There are three principle reactions of pyruvate.

First, under aerobic conditions, many micro-organisms will generate energy using the citric acid cycle and the conversion of pyruvate into acetyl coenzyme A, catalysed by pyruvate dehydrogenase (PDH).

Second, under anaerobic conditions, certain ethanologenic organisms can carry out alcoholic fermentation by the decarboxylation of pyruvate into acetaldehyde, catalysed by pyruvate decarboxylase (PDC) and the subsequent reduction of acetaldehyde into ethanol by NADH, catalysed by alcohol dehydrogenase (ADH).

A third process is the conversion of pyruvate into lactate which occurs through catalysis by lactate dehydrogenase (LDH).

There has been much interest in using micro-organisms for the production of ethanol using either micro-organisms that undergo anaerobic fermentation naturally or through the use of recombinant micro-organisms which incorporate the pyruvate decarboxylase and alcohol dehydrogenase genes. Although there has been some success in producing ethanol by using these micro-organisms, fermentation is often compromised by the increased concentration of the ethanol, especially where the micro-organism has a low level of ethanol tolerance.

Thermophilic bacteria have been proposed for ethanol production, and their use has the advantage that fermentation can be carried out at elevated temperatures which allows the ethanol produced to be removed as vapour at temperatures above 50° C.; this also permits fermentation to be carried out using high sugar concentrations. However, finding suitable thermophilic bacteria which can produce ethanol efficiently is problematic.

WO01/49865 discloses a Gram-positive bacterium which has been transformed with a heterologous gene encoding pyruvate decarboxylase and which has native alcohol dehydrogenase function, for the production of ethanol. The bacterium is a thermophilic Bacillus and the bacterium may be modified by the inactivation of the lactate dehydrogenase gene using transposon insertion. The bacteria disclosed in W001149865 are all derived from Bacillus Strain LLD-R, a sporulation-deficient strain that arose spontaneously from culture, and in which the Idh gene has been inactivated by spontaneous mutation or by chemical mutagenesis. Strains LN and TN are disclosed as improved derivatives of strain LLD-R. However, all strains contain a Hae III type restriction systems that impedes plasmid transformation and therefore prevents the transformation within un-methylated DNA.

WO01/85966 discloses microorganisms that are prepared by in vivo methylation to overcome the restriction problems. This requires transformation with Hae III methyltransferase from Haemophilus aegyptius into strains LLD-R, LN and TN. However, strains LLD-R, LN and TN are unstable mutants and spontaneously revert to lactate-producing wild-type strains, particularly at low pH and in high sugar concentrations. This results in fermentation product changes from ethanol to lactate, making the strains unsuitable for ethanol production.

WO02/29030 discloses that strain LLD-R and its derivatives include a naturally-occurring insertion element (1E) in the coding region of the Idh gene. Transposition of this into (and out of) the Idh gene and subsequent gene inactivation is unstable, resulting in reversion. The proposed solution to this was to integrate plasmid DNA into the IE sequence.

Therefore, in the art, the production of microorganisms for ethanol production relies on modifying laboratory-produced chemically mutated Bacillus microorganisms, treating these with in vivo methylation procedures and further modifying the microorganisms to integrate plasmid DNA into the IE sequence. The procedure is complex, uncertain and there are also regulatory issues on how the strains can be used.

There is therefore a need for improved microorganisms for ethanol production.

### SUMMARY OF THE INVENTION

According to a first aspect of the present invention, a thermophilic microorganism is modified to permit the increased production of ethanol, the modification being the inactivation of the lactate dehydrogenase gene of a wild-type thermophilic microorganism.

According to a second aspect of the present invention, a microorganism is that deposited as NCIMB Accession No. 41275.

According to a third aspect of the present invention, a method for the production of ethanol comprises culturing a microorganism according to the definition provided above under suitable conditions in the presence of a C₃, C₅ or C₆ sugar.

According to a fourth aspect of the present invention, a plasmid is that defined herein as pUB190-Idh (deposited as NCIMB Accession No. 41276).

According to a fifth aspect of the present invention, a thermophilic microorganism comprises the plasmid defined herein as pUB190 (FIG. 4).

### DESCRIPTION OF THE DRAWINGS

The present invention is described with reference to the accompanying figures, wherein:

FIG. 1 is a graph showing ethanol production of a microorganism of the invention (ldh 35) with different substrates;

FIG. 2 is a graph showing ethanol production of a microorganism of the invention (ldh 58) with different substrates;

FIG. 3 is a graph showing ethanol production by an LDH knockout mutant of 11955;

FIGS. 4 and 5 are schematic representations of the pUB plasmids utilised in the invention; FIG. 4 is the Idh mutant according to the invention; and

FIG. 6 is a graph showing stability of the LDH knockout mutant under different culture conditions.

### DESCRIPTION OF THE INVENTION

The present invention is based on the modification of a wild-type thermophilic microorganism to disrupt the expression of the lactate dehydrogenase gene.

Inactivating the lactate dehydrogenase gene helps to prevent the breakdown of pyruvate into lactate, and therefore promotes (under appropriate conditions) the breakdown of pyruvate into ethanol using pyruvate decarboxylase and alcohol dehydrogenase. It is preferred if the lactate dehydrogenase gene is disrupted by a deletion within or of the gene.

The wild-type microorganism may be any thermophilic microorganism, but it is preferred if the microorganism is of the Bacillus spp. In particular, it is preferred if the microorganism is of the Geobacillus species, in particular Geobacillus thermoglucosidasius.

The microorganisms selected for modification are said to be “wild-type”, i.e. they are not laboratory-produced mutants. The microorganisms may be isolated from environmental samples expected to contain thermophiles. Isolated wild-type microorganisms will have the ability to produce ethanol but, unmodified, lactate is likely to be the major fermentation product. The isolates are also selected for their ability to grow on hexose and/or pentose sugars, and oligomers thereof, at thermophilic temperatures.

It is preferable that the microorganism of the invention has certain desirable characteristics which permit the microorganism to be used in a fermentation process. The microorganism should preferably have no restriction system, thereby avoiding the need for in vivo methylation. In addition, the microorganism should be stable to at least 3% ethanol and should have the ability to utilise C₃, C₅ and C₆ sugars (or their oligomers) as a substrate, including cellobiose and starch. It is preferable if the microorganism is transformable at a high frequency. Furthermore, the microorganism should have a growth rate in continuous culture to support dilution rates of 0.3 h⁻¹ and above (typically 0.3 OD₆₀₀).

The microorganism will be a thermophile and will grow in the temperature range of 40° C.-85° C. Preferably, the microorganism will grow within the temperature range 50° C.-70° C. In addition, it is desirable that the microorganism grows in conditions of pH7.2 or below, in particular pH6.9-pH4.5.

The microorganism may be a spore-former or may not sporulate. The success of the fermentation process does not depend necessarily on the ability of the microorganism to sporulate, although in certain circumstances it may be preferable to have a sporulator, when it is desirable to use the microorganisms as an animal feed-stock at the end of the fermentation process. This is due to the ability of sporulators to provide a good immune stimulation when used as an animal feed-stock. Spore-forming microorganisms also have the ability to settle out during fermentation, and therefore can be isolated without the need for centrifugation. Accordingly, the microorganisms can be used in an animal feed-stock without the need for complicated or expensive separation procedures.

The nucleic acid sequence for lactate dehydrogenase is now known. Using this sequence, it is possible for the skilled person to target the lactate dehydrogenase gene to achieve inactivation of the gene through different mechanisms. It is preferred if the lactate dehydrogenase gene is inactivated either by the insertion of a transposon, or, preferably, by the deletion of the gene sequence or a portion of the gene sequence. Deletion is preferred, as this avoids the difficulty of reactivation of the gene sequence which is often experienced when transposon inactivation is used. In a preferred embodiment, the lactate dehydrogenase gene is inactivated by the integration of a temperature-sensitive plasmid (plasmid pUB190-Idh), which achieves natural homologous recombination or integration between the plasmid and the microorganism's chromosome. Chromosomal integrants can be selected for on the basis of their resistance to an antibacterial agent (for example, kanamycin). The integration into the lactate dehydrogenase gene may occur by a single cross-over recombination event or by a double (or more) cross-over recombination event.

In a preferred embodiment, the micro-organism comprises a heterologous alcohol dehydrogenase gene and a heterologous pyruvate decarboxylase gene. The expression of these heterologous genes results in the production of enzymes which redirect the metabolism so that ethanol is the primary fermentation product. These genes may be obtained from micro-organisms that typically undergo anaerobic fermentation, including zymomonas species, including zymomonas mobilis.

Methods for the preparation and incorporation of these genes into microorganisms are known, for example in Ingram et al, Biotech & BioEng, 1998; 58 (2+3): 204-214 and U.S. Pat. No. 5,916,787, the content of each being incorporated herein by reference. The genes may be introduced in a plasmid or integrated into the chromosome, as will be appreciated by the skilled person.

The microorganisms of the invention may be cultured under conventional culture conditions, depending on the thermophilic microorganism chosen. The choice of substrates, temperature, pH and other growth conditions can be selected based on known culture requirements, for example see WO01/49865 and WO01/85966, the content of each being incorporated herein by reference.

The present invention will now be described, by way of example only, with reference to the accompanying drawings, in the following examples.

### Inactivation of the LDH Gene

### EXAMPLE 1

### Single Crossover Mutation

### Development of the LDH Knockout Vector

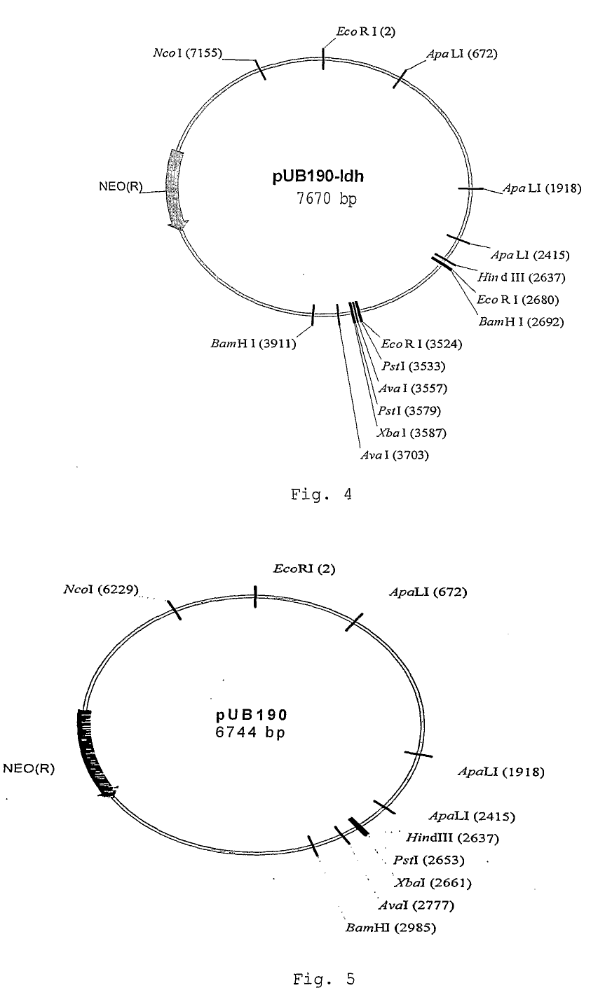

A partial Idh gene fragment of approx 800 bp was subcloned into the temperature sensitive delivery vector pUB190 (SEQ ID NO. 1) using HindIII and XbaI resulting in a 7.7 kb plasmid pUB190-Idh (FIG. 4 and SEQ ID NO. 2). Plasmid pUB190 has been deposited at NCIMB as indicated below. Ten putative E. coli JM109 (pUB190-Idh) transformants were verified by restriction analysis and two cultures used to produce plasmid DNA for transformation purposes. Digestion of pUB190-Idh with HindIII and XbaI releases the expected Idh fragment.

Transformation of Geobacillus thermoglucosidasius 11955 with pUB190-Idh

Transformants were obtained with all three plasmids tested after 24-48 hrs at 54° C. with kanamycin selection. The Idh transformants were purified from Geobacillus thermoglucosidasius (Gt) 11955 and verified by restriction analysis using HindIII and XbaI.

### LDH Gene Knockout

Gene knockout was performed by integration of a temperature-sensitive plasmid into the Idh gene on the chromosome.

Plasmid pUB190-Idh replicates at 54° C. in Gt11955 but not at 65° C. Selection was maintained with kanamycin (kan) (12 μg/ml). The growth temperature was then increased to 65° C. (the plasmid is no longer replicative). Natural recombination or integration occurs between the plasmid and chromosome. Chromosomal integrants were selected for by their resistance to kanamycin. Integration was directed towards the ldh gene since an homologous sequence resides on the plasmid. Targeted integration into the Idh gene occurred by a process known as single cross-over recombination. The plasmid becomes incorporated into the Idh gene resulting in an inactive Idh gene. Tandem repeats may occur if several copies of the plasmid integrate.

### Methodology and Results

Two different methods were attempted for integration:

Method 1: 4×50 ml TGP (kan) cultures were grown at 54° C. for 12-18 hours. The cells were pelleted by centrifugation and resuspended in 1 ml of TGP. The resuspension was plated (5×200 ml) on TGP (kan) plates and incubated overnight at 68° C. Integrants were picked and plated onto a 50-square grid on fresh TGP (kan) plates and incubated o/n at 68° C.

Method 2: 1×50 ml TGP (km) cultures was grown at 54° C. for 12-18 hours. 1 ml of the culture was used to inoculate 50 ml of fresh TGP (kan) cultures which was grown at 68° C. for 12-18 hours. This was sub-cultured the following day into 50 ml of fresh TGP (kan) cultures and grown at 68° C. for another 12-18 hours. The culture was plated out on TGP (km) plates and incubated at 68° C. overnight. Confluent growth was obtained on the plates. Single colonies were plated onto a 50-square grid on fresh TGP (kan) plates and incubated overnight at 68° C.

### Screening

The putative integrants were screened for Idh gene knockout using the following:

1) A plate screen

Replica plating of several hundred integrants onto SAM2 plates (with kan) at 68° C. Lactate negative cells produce less acid and may have a growth advantage over the wild type on fermentative media without buffers.

### 2) A PCR Screen

Colony PCR was used to determine whether the plasmid has integrated into the ldh gene. By choosing primers that flank the integration site, it was possible to determine whether Idh gene integration had occurred (no PCR fragment was amplified for inserts).

3) Lactate Assay

This assay determines whether the integrants produce lactate when grown overnight in SAM2 (kan) at 68° C. The culture supernatant was tested for the concentration of lactate with the Sigma lactate reagent for lactae determination. Lactate negative integrants were further characterised by PCR and evaluated in a fermenter for stability.

Electroporation Protocol for Geobacillus thermoqlucosidasius NCIMB 11955

A frozen stock of NCIMB 11955 was made by growing an overnight culture in TGP medium (250 rpm at 55° C., 50 ml volume in a 250 ml conical flask, OD₆₀₀), adding an equal volume of 20% glycerol and dividing into 1 ml aliqouts and storing in cryotubes at −80° C. 1 ml of this stock was used to inoculate 50 ml of TGP in a 250 ml conical flask, incubated at 55° C., 250 rpm, until the culture reaches OD₆₀₀ 1.4.

The flask was cooled on ice for 10 minutes, then the culture centrifuged for 20 minutes at 4000 rpm at 4° C. The pellet was resuspended in 50 ml ice-cold electroporation medium and centrifuged at 4,000 rpm for 20 minutes. Three further washes were carried out, 1×25 ml and 2×10 ml, and then the pellet resuspended in 1.5 ml ice-cold electroporation medium and divided into 64 μl aliquots.

For the electroporation, 1-2 μl of DNA was added to 60 μl of electrocompetent cells in an eppendorf tube kept on ice, and gently mixed. This suspension was transferred to a pre-cooled electroporation cuvette (1 mm gap) and electroporated at 2500V, 10 μF capacitance and 600Ω resistance.

Immediately after the pulse, 1 ml TGP was added, mixed, and the suspension transferred to a screw top tube and incubated at 52° C. for 1 hour in a shaking waterbath. After incubation the suspension was either plated directly (eg 2×0.5 ml) or centrifuged at 4,000 rpm for 20 minutes, resuspended in 200 μl-500 μl TGP, and plated on TGP agar containing the appropriate antibiotic.

| Electroporation medium | TGP medium | | |
|---|---|---|---|
| 0.5M sorbitol | Tryptone | 17 | g/L |
| 0.5M mannitol | Soy peptone | 3 | g/L |
| 10% glycerol | K₂HPO₄ | 2.5 | g/L |
| | NaCl | 5 | g/L |
| | pH | to 7.3 | |
| | Additions post-autoclaving; | | |
| | Sodium pyruvate | 4 | g/l |
| | Glycerol | 4 | ml/L |

### Inactivation of the LDH Gene.

### Double Cross-Over Mutation

Primers were designed based on the available 11955 LDH sequence. The knock-out strategy was based on the generation of internal deletions within the LDH gene by two approaches.

In strategy 1, two existing unique restriction sites near the middle of the LDH coding sequence were exploited to generate a deletion. A single large pcr product was generated from genomic DNA covering most of the available LDH sequence, and cloned into the SmaI site in the multiple cloning site of pUC19. The pUC19 clone was then digested sequentially with BstEII and BsrGI and religated after Klenow digestion, to generate an internal deletion in the LDH gene between BstEII and BsrGI.

In strategy 2 (see Example 2) the LDH gene was cloned as 2 PCR products, introducing NotI sites on the oligo primers to allow the 2 PCR products to be ligated together in pUC19, with the generation of a deletion in the middle of the LDH sequence. The two LDH genes with the internal deletions were subcloned into three potential delivery systems for Geobacillus.

| Plasmid | Details |
|---|---|
| pCU1 | 4.94 kb, shuttle vector based on pC194, carries cat & bla |
| pBT2 | 6.97 kb, shuttle vector derived from a temperature sensitive |
| | mutant of pE194, carries cat and bla. |
| pTVOmcs | 4.392 kb, derived from pE194ts, carries cat (no Gram |
| | negative replicon). |

The delivery vectors were transformed into 11955 by electroporation.

### Genetic Strategy Information: Development of Delivery Systems for Homologous Recombination.

To generate knockouts, an efficient system is required to deliver a mutated gene into the target organism and select for integration into the genome by homologous recombination with the target “wild-type” gene. In principle, this could be achieved by introducing the DNA on an E. coli vector without a Gram-positive replicon but which carries a Gram-positive selectable marker. This requires a high transformation efficiency. The electroporation method developed for Geobacillus 11955 generates 3×10⁴ transformants per μg of DNA with pNW33N. The Gram-positive replicon is derived from pBC1 in the BGSC catalogue, and from pTHT15 in the sequence database.

The cat gene on pNW33N is used for selection in both E. coli and Geobacillus. Temperature-sensitive mutants of pNW33N were generated by passaging the plasmid through the Statagene XL1r ed mutator strain.

### EXAMPLE 2

### Generation of an LDH Mutant by Gene Replacement

A further strategy was designed to generate a stable mutation of the LDH gene in Geobacillus thermoglucosidasius NCIMB 11955 by gene replacement. This strategy involved the generation of a 42 bp deletion close to the middle of the coding sequence, and the insertion at this position of 7 bp introducing a novel NotI restriction site. This inserted sequence was intended to cause a frame-shift mutation downstream. The strategy involved generating the deletion using 2 PCR fragments using oligo primers introducing the novel NotI site. Primers were designed based on the partial sequence of the LDH coding region from 11955. The sequence of the primers used is shown below.

Fragment 1:

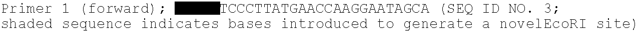

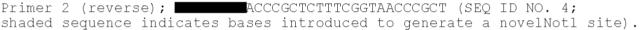

Fragment 2:

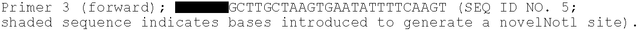

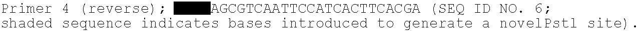

### Preparation of Genomic DNA.

Genomic DNA was prepared from 11955 to serve as template for PCR. Cells from a 20 ml overnight culture of 11955 grown in TGP medium at 52° C. were collected by centrifugation at 4,000 rpm for 20 mins. The cell pellet was resuspended in 5 ml of STE buffer 0.3M sucrose, 25 mM Tris HCl and 25 mM EDTA, adjusted to pH 8.0 containing 2.5 mg of lysozyme and 50 μl of 1 mg/ml ribonuclease A. This was incubated for 1 hour at 30° C., then 5 mg of proteinase K was added and 50 μl of 10% SDS followed by incubation at 37° C. for 1 hour. The lysed culture was then extracted sequentially with equal volumes of phenol/chloroform followed by chloroform before precipitation with isopropanol. After washing twice with ice-cold 70% ethanol, the DNA pellet was redissolved in 0.5 ml TE buffer.

### Generation of LDH Deletion Construct

PCR was carried out using a Robocycler Gradient 96 (Stratagene) and the reaction conditions were as follows: Cycle 1; denaturation at 95° C. for 5 min, annealing at 47° C. for 1 min, extension at 72° C. for 2 min, Cycle 2-30; denaturation at 95° C. for 1 min, annealing at 47° C. for 1 min, extension at 72° C. for 2 min, and a further incubation at 72° C. for 5 min. The enzymes used were an equal mixture of Pfu polymerase (Promega) and Taq polymerase (New England Biolabs, NEB). The buffer and dNTPs composition and concentration used was that recommended for Pfu by the suppliers. The PCR products obtained using genomic DNA from NCIMB 11955 as template were purified by agarose gel electrophoresis and eluted from the agarose gel by using the QIAquick Gel Extraction Kit (Qiagen). The purified PCR products were ligated to pUC19 (New England Biolabs) previously digested with SmaI and the ligation mixture was used to transform Escherichia coli DH10B (Invitrogen). Ampicillin-resistant colonies were selected and the contained plasmids were isolated and characterised by restriction analysis, and the orientation of the inserts was established.

A plasmid (pTM002) with fragment 2 inserted into pUC19 (with the novel PstI site introduced on Primer 4 closest to the PstI site in the multiple cloning site (mcs) of pUC19) was digested with NotI and PstI. The resulting fragment (approximately 0.4 kb) was ligated into a pUC19 plasmid (pTM001) bearing fragment 1 (with the novel EcoRI site introduced on Primer 1 closest to the EcoRI site in the mcs of pUC19) digested with NotI and PstI to linearise the plasmid. The ligation mixture was used to transform E. coli DH10B. Ampicillin resistant colonies were selected and the contained plasmids were isolated and characterised by restriction analysis. A plasmid (pTM003) with the expected restriction pattern for the desired construct (the LDH coding region carrying the deletion and introduced NotI site) was identified and verified by sequencing using M13mp18 reverse and forward primers.

The mutated LDH gene was excised from pTM003 by digestion with HindIII and EcoRI and purified by agarose gel electrophoresis followed by elution from the agarose gel using the QIAquick Gel Extraction Kit (as an approximately 0.8 kb fragment). This fragment was treated with Klenow polymerase (NEB, according to manufacturers instructions) to generate blunt ends and introduced into the pUB190 vector. This was achieved by blunt-end ligation with pUB190 linearised by digestion with XbaI and then Klenow-treated followed by gel-purification as before. The ligation mixture was used to transform E. coli SCS110 (Stratagene). Ampicillin-resistant colonies were selected and the contained plasmids were isolated and characterised by restriction analysis. A plasmid (pTM014) with the expected restriction pattern for the desired construct was identified and used to transform NCIMB 11955 by electroporation using the electroporation protocol as described in Example 1.

### Generation and Characterization of a Gene-Replacement LDH Mutant by Double-Crossover.

A presumptive primary integrant of pTM014 obtained in this fashion (strain TM15) was used to obtain double recombinants (gene replacement). This was achieved by serial sub-culture of TM15 in TGP medium without kanamycin. Five successive shaken cultures were used, alternating between 8 hours at 54° C. and 16 hours at 52° C., using 5 ml TGP in 50 ml tubes (Falcon) at 250 rpm, 1% transfer at each stage. After these 5 passages, the resulting culture was serially diluted in TGP and 100 μl samples plated on TGP agar plates for incubation at 54° C. Replica-plating of the resultant colonies onto TGP agar containing 12 μg/ml kanamycin was used to identify kanamycin-sensitive colonies. After streaking to single colonies on agar to purify, these kanamycin sensitive derivatives were tested for lactate production, and as expected, proved a mixture of LDH⁺ and LDH⁻. One LDH⁻ derivative, TM89, was further characterized by PCR and Southern blots.

Genomic DNA was prepared from TM15 (primary integrant) and TM89 (presumptive double recombinant LDH⁻), and used as template for PCR using Primers 1 and 4, using the conditions described above. Genomic DNA from 11955 was used as control. The PCR products (approx. 0.8 kb bands were obtained from all 3 templates) were purified by agarose gel electrophoresis and eluted from the agarose gel using the QIAquick Gel Extraction Kit. Samples were digested with Not I and run on a 0.7% agarose gel to visualize products. The PCR product of 11955 showed no evidence of NotI digestion, as expected, whereas the PCR product of TM89 gave 2 bands of around 0.4 kb, indicating the replacement of the wild-type gene with the mutated allele. NotI digestion of the PCR product of TM15, the primary integrant, gave predominantly the 2 bands seen with TM89, with a trace of the uncut (0.8 kb) band. This can be explained by the result obtained with Southern blotting of the TM15 genomic DNA.

Genomic DNA of 11955, TM15 and TM89 was digested with NotI, PstI and NotI, and HindIII and NotI, and subjected to agarose gel electrophoresis. The DNA was transferred onto a positively-charged nylon membrane (Roche) and hybridized with a DIG-labelled probe generated by PCR of the 11955 LDH gene using Primers 1 and 4 with DIG-labeled dNTps, following the suppliers instructions (Roche Molecular Biochemicals DIG application manual). The hybridizing bands were visualized using the detection kit supplied (Roche). The Southern blot showed evidence of a much-amplified band of approx. 7.5 kb in the NotI digest of TM15, with similarly-amplified bands of approx. 7 and 0.4 kb in the HindIII/NotI and PstI/NotI digests of TM15, indicating integration of multiple tandem copies of pTM014 integrated at the LDH locus in this primary integrant. With all 3 restriction digests, TM89 showed evidence of a different restriction pattern showing an extra hybridizing band compared to 11955, consistent with gene replacement. TM89 has been deposited at NCIMB under Accession No. 41275 as indicated below.

### EXAMPLE 3

### Ethanol Production by the Wild-Type Thermophile

Reproducible growth and product formation was achieved in fed-batch and continuous cultures for the wild-type thermophile. Tables 1, 2 and 3 show the conditions used in the fermentation process.

TABLE 1

| | Vol./L | | Final Conc. | |
|---|---|---|---|---|
| Chemical | | | | |
| NaH₂PO_{4•}2H₂O | | | 25 | mM |
| K₂SO₄ | | | 10 | mM |
| Citric acid. H₂O | | | 2 | mM |
| MgSO_{4•}7H₂O | | | 1.25 | mM |
| CaCl_{2•}2H₂O | | | 0.02 | mM |
| Sulphate TE Solution | 5 | ml | See below | |
| Na₂MoO_{4•}2H₂O | | | 1.65 | μM |
| Yeast Extract | 10 | g | | |
| Antifoam | 0.5 | ml | | |
| Post-auto addns: | | | | |
| 4M Urea | 25 | ml | 100 | mM |
| 1% Biotin | 300 | μl | 12 | μM |

| | | | |
|---|---|---|---|
| 20% Glucose² | 50 | ml | 1% |

TABLE 2
Sulphate Trace Elements Stock Solution

| | | | Medium | |
|---|---|---|---|---|
| Chemical | gl⁻¹ (ml) | | Conc. | |
| Conc. H₂SO₄ | 5 | ml | | |
| ZnSO₄•7H₂O | 1.44 | | 25 | μM |
| FeSO₄•7H₂O | 5.56 | | 100 | μM |
| MnSO₄•H₂O | 1.69 | | 50 | μM |
| CuSO₄•5H₂O | 0.25 | | 5 | μM |
| CoSO₄•7H₂O | 0.562 | | 10 | μM |
| NiSO₄•6H₂O | 0.886 | | 16.85 | μM |
| H₃BO₃ | 0.08 | | | |
| Deionised H₂O (final | 1000 | ml | | |
| vol.) | | | | |

TABLE 3
Fermenter Conditions

| | |
|---|---|
| Inoculum | 10% v/v |
| Volume | 1000 ml |
| Temperature | 60° C. |
| PH | 7.0 controlled with NaOH |
| Aeration | 0.4 vvm |
| N₂ flow | 0.05 lpm |
| Agitation | 400 rpm |
| Media | Urea Sulphates for Fermenters |
| Sugar feed | 100 ml 50% glucose |

TABLE 4
Summary of improvements in growth of wild-type Bacillus in
Batch Culture

| | Total Sugar | | mM | | | | |
|---|---|---|---|---|---|---|---|
| Media | added mM | OD600 | pyr | lact | form | acet | ethanol |
| Xylose | 603 | 8.5 | 9 | 187 | 5 | 75 | 22 |
| Glucose 1 | 504 | 4 | 2 | 295 | 36 | 28 | 39 |
| Glucose 2 | 504 | 3.7 | 19 | 322 | 111 | 55 | 123 |

### EXAMPLE 4

### Increasing Ethanol Production by Thermophiles

In order to achieve the target ethanol yields and productivity from the thermophile, lactic acid production was minimised through the knock-out of L-Lactate Dehydrogenase (LDH) activity by inactivation of the Idh gene. There were two approaches taken to inactivate the Idh gene: a single crossover recombination of marker DNA into the Idh region of the chromosome, preventing its transcription, or a double recombination of homologous regions of DNA into the Idh gene to create a mutation within the gene region, rendering it non-functional.

The single cross-over approach rapidly generated LDH-negative mutants which showed an increase in ethanol production when compared to the wild-type strain (NCIMB 11955).

### Improvements in Ethanol Production by the LDH Mutants

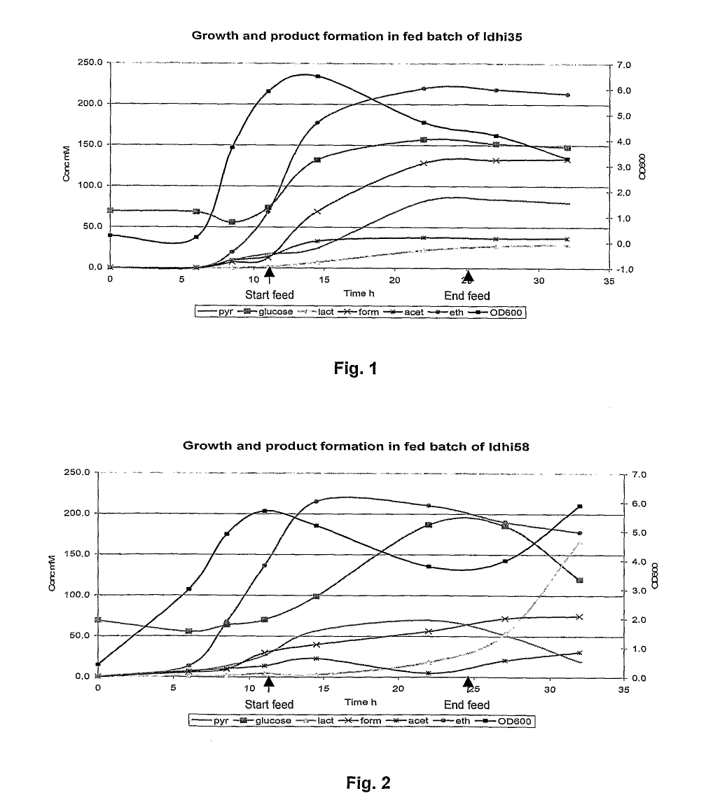

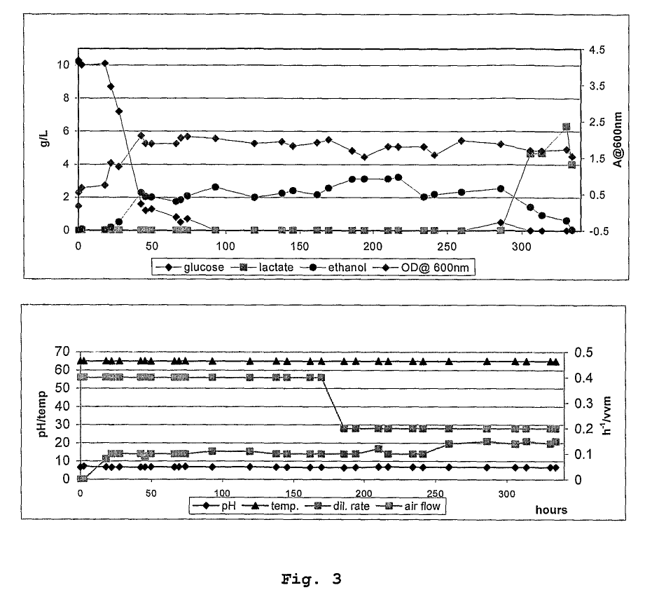

The LDH-negative mutants were grown in fed-batch cultures, in the established minimal media to measure the increase in ethanol production resulting from the knock-out of LDH activity. The change in the metabolite profiles of the LDH mutants are shown in Table 5, compared to the optimum ethanol production from the wild-type strain. Metabolite production profiles of two of the LDH mutants are shown in FIG. 1 and 2. Table 6 shows the increase of ethanol production caused by the knock-out of LDH activity.

TABLE 5
Summary of increase in ethanol production achieved by lactate mutants

| Organ- | Carbon | Total | | mM | | | | |
|---|---|---|---|---|---|---|---|---|
| ism | Source | Sugar | OD600 | pyr | lact | form | acet | ethanol |
| wild type | gluc | 603 | 3.7 | 19 | 322 | 111 | 55 | 123 |
| ldh 35 | gluc | 336 | 4.7 | 82 | 22 | 128 | 37 | 220 |
| ldh 58 | gluc | 336 | 5.2 | 57 | 3 | 40 | 23 | 215 |

TABLE 6
Increased ethanol production by LDH mutants in fed-batch culture

| | | | Glucose | | | Lactate | Lactate | | | Ethanol |
|---|---|---|---|---|---|---|---|---|---|---|
| | Carbon | | in | Residual | | g/g | g/g | | Ethanol | g/g |
| Organism | Source | OD600 | feed g | glu g | Lactate g | cells | glu | Ethanol g | g/g/cells | glu |
| wild type | gluc | 3.7 | 60.48 | 1.62 | 37.89 | 48.89 | 0.64 | 4.05 | 5.22 | 0.07 |
| ldh 35 | gluc | 4.7 | 60.48 | 17.80 | 1.98 | 1.36 | 0.05 | 10.12 | 6.95 | 0.24 |
| ldh 58 | gluc | 5.2 | 60.48 | 28.26 | 0.27 | 0.17 | 0.01 | 9.89 | 6.14 | 0.31 |

The LDH mutants produced significantly higher yields of ethanol than the wild-type thermophile, demonstrating the successful rerouting of the metabolism of these thermophiles. Further optimisation of culture conditions and media constituents for the LDH mutant strains will result in increased ethanol yields.

### Ethanol Production by the LDH Mutants in Continuous Culture

The most stable of the single crossover lactate mutants was grown in continuous culture to assess its long term stability as an ethanol producer. The strain was cultured in minimal media with glucose as a carbon source. Steady state culture was observed for approximately 290 hours before reversion to lactate production occurred.

### EXAMPLE 5

The double crossover mutant TM89 (Example 2) was assayed for ethanol production as set out in Example 4. The results are shown in Table 8 and show that the mutant achieved significant levels of ethanol production (greater than 200 mM). The mutant was also assayed for ethanol production utilising glucose or xylose as the carbon source. The results are shown in Table 7.

TABLE 7

| | | Total | Sugar | | | | | | |
|---|---|---|---|---|---|---|---|---|---|
| | Carbon | sugar/ | residue/ | | Concentration/mM | | | | |
| Organism | source | mM | mM | OD₆₀₀ | ethanol | lactate | pyruvate | acetate | formate |
| WT | Glucose | 794 | 130 | 5.3 | 46 | 279 | 7 | 69 | 9 |
| WT | Xylose | 953 | 122 | 8.5 | 22 | 187 | 9 | 75 | 5 |
| TM89 | Glucose | 862 | 53 | 6.5 | 214 | 5* | 109 | 10 | 88 |
| TM89 | Xylose | 1045 | 142 | 4.9 | 130 | 14* | 24 | 36 | 39 |

Glucose was shown to be the best carbon source, but the results clearly show that the organisms are able to ferment xylose without further modification.

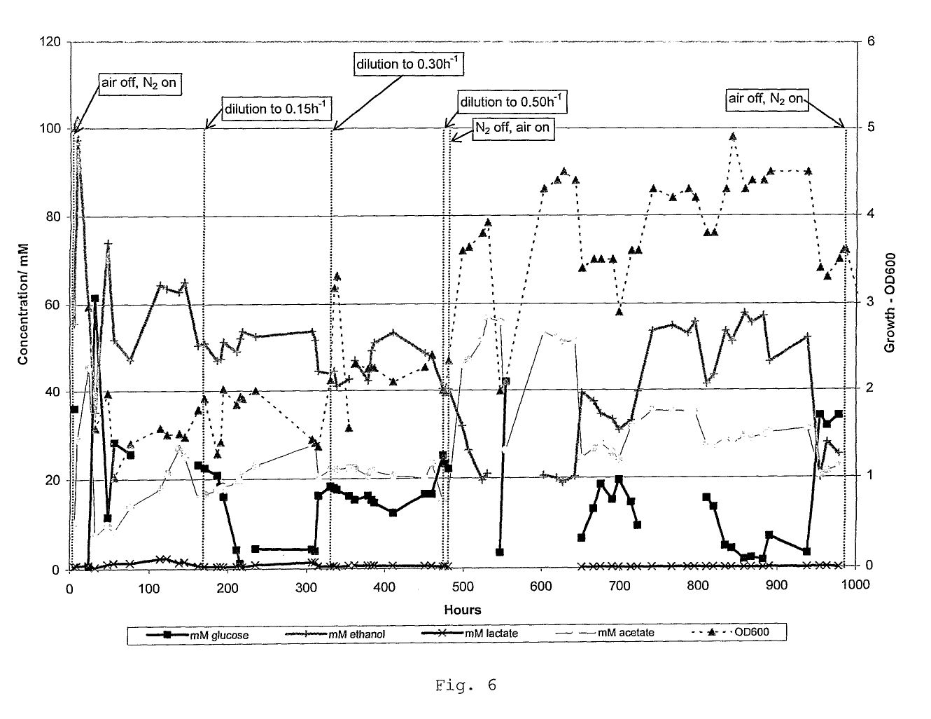

The stability of the lactate dehydrogenase-negative mutant was also tested under a variety of conditions in continuous culture over a period of 1500 hours. The results are shown in FIG. 6, and demonstrate that the lactate dehydrogenase-negative phenotype remains stable, despite significant challenges to the culture, and does not rely on antibiotic selection to remain. During this continuous run the dilution rate was varied between 0.1 hr⁻¹ and 0.5 hr⁻¹ and the culture was switched between oxic (air on) and anoxic (N₂ on) conditions with no change in the lactate concentration. More significantly the pH of the culture was varied and dropped to pH 4.4, which is outside the normal pH range for growth of the wild type organism. However, the culture quickly recovered with no change in phenotype, namely the lactate concentration did not change.

The microorganism defined herein as TM89 and the plasmid pUB 190-Idh have been deposited under NCIMB Accession Nos. 41275 and 41276, respectively. The depository is: NCIMB Ltd, Ferguson Building, Craibstone Estate, Bucksburn, Aberdeen, AB21 9YA, United Kingdom.

TABLE 8

| | | | | | Sugar | | | | | | | | | |
|---|---|---|---|---|---|---|---|---|---|---|---|---|---|---|
| | Continuous | Air | | Biomass | used | g/L | | g/g sugar | | | g/g biomass | | g/L/h | |
| Organism | Feed rate | WM | Sugar | g/L | g/L | lact | ethanol | biomass | lact | ethanol | lact | ethanol | lact | ethanol |
| wild type | D = 0.04 | 0.06 | gluc 2% | 0.68 | 20.00 | 11.25 | 1.56 | 0.03 | 0.56 | 0.08 | 16.50 | 2.29 | 0.45 | 0.06 |
| wild type | D = 0.09 | 0.06 | gluc 2% | 1.02 | 20.00 | 7.29 | 1.79 | 0.05 | 0.36 | 0.09 | 7.13 | 1.75 | 0.66 | 0.16 |
| Ldh-21 | D = 0.1 | 0.02 | gluc 2% | 0.51 | 10.00 | 0 | 3.15 | 0.05 | 0.00 | 0.32 | 0.00 | 6.18 | 0.00 | 0.32 |

We claim:

1. A thermophilic microorganism, modified to permit the increased production of ethanol, wherein the modification is the inactivation of the lactate dehydrogenase gene of a wild-type thermophilic microorganism.

2. The microorganism according to claim 1, wherein the microorganism does not comprise a restriction system.

3. The microorganism according to claim 1, wherein the microorganism is geobacillus species.

4. The microorganism according to claim 1, wherein the microorganism is geobacillus thermoglucosidasius.

5. The microorganism according to claim 1, wherein the microorganism is a spore-former.

6. The microorganism according to claim 1, wherein the microorganism is stable in a culture medium comprising up to 30% (w/v) ethanol.

7. The microorganism according to claim 1, wherein the microorganism can metabolise cellobiose and/or starch, or oligomer thereof.

8. The microorganism according to claim 1, wherein the microorganism is transformable at high frequency.

9. The microorganism according to claim 1, wherein the microorganism grows at a temperature from 40° C.-85° C., preferably 50° C.-70° C.

10. The microorganism according to claim 1, wherein the microorganism comprises a non-native pdc gene.

11. The microorganism according to claim 1, wherein the microorganism comprises a non-native adh gene.

12. The microorganism according to claim 1, wherein the microorganism does not comprise an integration element in the lactate dehydrogenase gene.

13. A microorganism deposited as NCIMP Accession No. 41275

14. A method for the production of ethanol comprising culturing a microorganism according to any preceding claim under suitable conditions in the presence of a C₃, C₅ or C₆ sugar, or oligomer thereof.

15. The method according to claim 14, wherein the method is carried out at a temperature of between 40° C.-70° C.

16. The method according to claim 15, wherein the temperature is from 52° C.-65° C.

17. The method according to claim 14, wherein the microorganism is maintained in a culture at a pH of between 4 and 7.5.

18. A plasmid defined herein as pUB 190-Idh.

19. A thermophilic microorganism, comprising the plasmid of claim 18.

20. An animal feed, comprising the microorganism according to claim 1.


SEQUENCE LISTING (6 sequences)

SEQ ID NO: 1 - DNA, 6744 bp, Geobacillus thermoglucosidasius
gaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttgcatgcctgcaggtcgactctagaaaatcaatctctttttccaaagtttgttttttaaatttagctgtctcaatatgtttacggtcagagccacgttcaccacgcttcaactcaaaaccctgttttttcatatgctcggggaatttatcttgtagccataacagttcttgacgattaaacacattttttccttgcagttttccatcacgcataggcacaacacctaaatgcatgtgaggggtttgctcatcattatgaactgttgcataagcaatattttgcttgccatatcgttcggaaaataatttataactttcctcaaaaaatcgtttttgttctcctggatccagttgctcaaaaaaatctcggtcagatgttactagcaactcatttacaagaacagcatctttcctcgtttttcttgtacctgttttttgtgattcaataatttctttgacacgttcgttgtaatcaatatttttatcatttttcaaatcataattttcacgtgttcgctcatggtcaatatcatcattcgttctactttttcgctctctttgattatgaaattgcatgccttttagtccagctgatttcactttttgcattctacaaactgcataactcatatgtaaatcgctcctttttaggtggcacaaatgtgaggcattttcgctctttccggcaaccacttccaagtaaagtataacacactatactttatattcataaagtgtgtgctctgcgaggctgtcggcagtgccgaccaaaaccataaaacctttaagacctttcttttttttacgagaaaaaagaaacaaaaaaacctgccctctgccacctcagcaaaggggggttttgctctcgtgctcgtttaaaaatcagcaagggacaggtagtattttttgagaagatcactcaaaaaatctccacctttaaacccttgccaatttttattttgtccgttttgtctagcttaccgaaagccagactcagcaagaataaaatttttattgtctttcggttttctagtgtaacggacaaaaccactcaaaataaaaaagatacaagagaggtctctcgtatcttttattcagcaatcgcgcccgattgctgaacagattaataatagattttagctttttatttgttgaaaaaagctaatcaaattgttgtcgggatcaattactgcaaagtctcgttcatcccaccactgatcttttaatgatgtattggggtgcaaaatgcccaaaggcttaatatgttgatataattcatcaattccctctacttcaatgcggcaactagcagtaccagcaataaacgactccgcacctgtacaaaccggtgaatcattactacgagagcgccagccttcatcacttgcctcccatagatgaatccgaacctcattacacattagaactgcgaatccatcttcatggtgaaccaaagtgaaacctagtttatcgcaataaaaacctatactctttttaatatccccgactggcaatgccgggatagactgtaacattctcacgcataaaatcccctttcattttctaatgtaaatctattaccttattattaattcaattcgctcataattaatcctttttcttattacgcaaaatggcccgatttaagcacaccctttattccgttaatgcgccatgacagccatgataattactaatactaggagaagttaataaatacgtaaccaacatgattaacaattattagaggtcatcgttcaaaatggtatgcgttttgacacatccactatatatccgtgtcgttctgtccactcctgaatcccattccagaaattctctagcgattccagaagtttctcagagtcggaaagttgaccagacattacgaactggcacagatggtcataacctgaaggaagatctgattgcttaactgcttcagttaagaccgaagcgctcgtcgtataacagatgcgatgatgcagaccaatcaacatggcacctgccattgctacctgtacagtcaaggatggtagaaatgttgtcggtccttgcacacgaatattacgccatttgcctgcatattcaaacagctcttctacgataagggcacaaatcgcatcgtggaacgtttgggcttctaccgatttagcagtttgatacactttctctaagtatccacctgaatcataaatcggcaaaatagagaaaaattgaccatgtgtaagcggccaatctgattccacctgagatgcataatctagtagaatctcttcgctatcaaaattcacttccaccttccactcaccggttgtccattcatggctgaactctgcttcctctgttgacatgacacacatcatctcaatatccgaatagggcccatcagtctgacgaccaagagagccataaacaccaatagccttaacatcatccccatatttatccaatattcgttccttaatttcatgaacaatcttcattctttcttctctagtcattattattggtccattcactattctcattcccttttcagataattttagatttgcttttctaaataagaatatttggagagcaccgttcttattcagctattaataactcgtcttcctaagcatccttcaatccttttaataacaattatagcatctaatcttcaacaaactggcccgtttgttgaactactctttaataaaataatttttccgttcccaattccacattgcaataatagaaaatccatcttcatcggctttttcgtcatcatctgtatgaatcaaatcgccttcttctgtgtcatcaaggtttaattttttatgtatttcttttaacaaaccaccataggagattaaccttttacggtgtaaaccttcctccaaatcagacaaacgtttcaaattcttttcttcatcatcggtcataaaatccgtatcctttacaggatattttgcagtttcgtcaattgccgattgtatatccgatttatatttatttttcggtcgaatcatttgaacttttacatttggatcatagtctaatttcattgcctttttccaaaattgaatccattgtttttgattcacgtagttttctgtattcttaaaataagttggttccacacataccaatacatgcatgtgctgattataagaattatctttattatttattgtcacttccgttgcacgcataaaaccaacaagatttttattaatttttttatattgcatcattcggcgaaatccttgagccatatctgacaaactcttatttaattcttcgccatcataaacatttttaactgttaatgtgagaaacaaccaacgaactgttggcttttgtttaataacttcagcaacaaccttttgtgactgaatgccatgtttcattgctctcctccagttgcacattggacaaagcctggatttacaaaaccacactcgatacaactttctttcgcctgtttcacgattttgtttatactctaatatttcagcacaatcttttactctttcagcctttttaaattcaagaatatgcagaagttcaaagtaatcaacattagcgattttcttttctctccatggtctcacttttccactttttgtcttgtccactaaaacccttgatttttcatctgaataaatgctactattaggacacataatattaaaagaaacccccatctatttagttatttgtttagtcacttataactttaacagatggggtttttctgtgcaaccaattttaagggttttcaatactttaaaacacatacataccaacacttcaacgcacctttcagcaactaaaataaaaatgacgttatttctatatgtatcaagataagaaagaacaagttcaaaaccatcaaaaaaagacaccttttcaggtgctttttttattttataaactcattccctgatctcgacttcgttctttttttacctctcggttatgagttagttcaaattcgttctttttaggttctaaatcgtgtttttcttggaattgtgctgttttatcctttaccttgtctacaaaccccttaaaaacgtttttaaaggcttttaagccgtctgtacgttccttaag

SEQ ID NO: 2 - DNA, 7673 bp, Geobacillus thermoglucosidasius
gaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttggtaccgagctcactagtcacggccgccagtgtgctggaattcgcccttggatcctttgcccttatgaaccaaggaatagcagatgagttagtattgattgatgtaaataagaataaggcagagggcgatgtgatggatttaaatcacggaaaagtattcgcgccgaagccgatgaatatttggtttggagattatcaagattgccaagacgccgatttggtggtgatttgtgcaggggctaaccaaaagccgggagaaacaagactggatcttgttgacaaaaatattaatatcttcaaaacgattgtcgattctgtgatgaaatccggatttgatggcgtttttcttgtggcaacgaacccagtggatattttaacgtatgctacttggaaatttagcgggttaccgaaagagcgggtaatcggctcaggaacgattcttgatacagcaagattccgcttcttgctaagtgaatattttcaagtggctccgaccaatgtacatgcgtatattattggcgagcatggggatacagagctgcctgtttggagccatgcggaaattggaagcattccagttgagcaaatattgatgcaaaacgataactatagaaaagaggatttagacaatatctttgttaatgttcgtgatgcggcatatcaaatcattgagaaaaaaggggcaacgtattacggcattgcaatgggattagtccgtatcactcgtgctattttgcacaatgaaaatgccatcttaaccgtttctgctcatttggacggccaatatggcgaacgaaatgtttatattggcgtgcctgccattatcaaccgaaacggtattcgtgaagtgatggaattgacgctaaatgaaacagaacaagggcgaattctgcagatatccatcacactggcggccgctcgagcatgcagcatgcctgcaggtcgactctagaaaatcaatctctttttccaaagtttgttttttaaatttagctgtctcaatatgtttacggtcagagccacgttcaccacgcttcaactcaaaaccctgttttttcatatgctcggggaatttatcttgtagccataacagttcttgacgattaaacacattttttccttgcagttttccatcacgcataggcacaacacctaaatgcatgtgaggggtttgctcatcattatgaactgttgcataagcaatattttgcttgccatatcgttcggaaaataatttataactttcctcaaaaaatcgtttttgttctcctggatccagttgctcaaaaaaatctcggtcagatgttactagcaactcatttacaagaacagcatctttcctcgtttttcttgtacctgttttttgtgattcaataatttctttgacacgttcgttgtaatcaatatttttatcatttttcaaatcataattttcacgtgttcgctcatggtcaatatcatcattcgttctactttttcgctctctttgattatgaaattgcatgccttttagtccagctgatttcactttttgcattctacaaactgcataactcatatgtaaatcgctcctttttaggtggcacaaatgtgaggcattttcgctctttccggcaaccacttccaagtaaagtataacacactatactttatattcataaagtgtgtgctctgcgaggctgtcggcagtgccgaccaaaaccataaaacctttaagacctttcttttttttacgagaaaaaagaaacaaaaaaacctgccctctgccacctcagcaaaggggggttttgctctcgtgctcgtttaaaaatcagcaagggacaggtagtattttttgagaagatcactcaaaaaatctccacctttaaacccttgccaatttttattttgtccgttttgtctagcttaccgaaagccagactcagcaagaataaaatttttattgtctttcggttttctagtgtaacggacaaaaccactcaaaataaaaaagatacaagagaggtctctcgtatcttttattcagcaatcgcgcccgattgctgaacagattaataatagattttagctttttatttgttgaaaaaagctaatcaaattgttgtcgggatcaattactgcaaagtctcgttcatcccaccactgatcttttaatgatgtattggggtgcaaaatgcccaaaggcttaatatgttgatataattcatcaattccctctacttcaatgcggcaactagcagtaccagcaataaacgactccgcacctgtacaaaccggtgaatcattactacgagagcgccagccttcatcacttgcctcccatagatgaatccgaacctcattacacattagaactgcgaatccatcttcatggtgaaccaaagtgaaacctagtttatcgcaataaaaacctatactctttttaatatccccgactggcaatgccgggatagactgtaacattctcacgcataaaatcccctttcattttctaatgtaaatctattaccttattattaattcaattcgctcataattaatcctttttcttattacgcaaaatggcccgatttaagcacaccctttattccgttaatgcgccatgacagccatgataattactaatactaggagaagttaataaatacgtaaccaacatgattaacaattattagaggtcatcgttcaaaatggtatgcgttttgacacatccactatatatccgtgtcgttctgtccactcctgaatcccattccagaaattctctagcgattccagaagtttctcagagtcggaaagttgaccagacattacgaactggcacagatggtcataacctgaaggaagatctgattgcttaactgcttcagttaagaccgaagcgctcgtcgtataacagatgcgatgatgcagaccaatcaacatggcacctgccattgctacctgtacagtcaaggatggtagaaatgttgtcggtccttgcacacgaatattacgccatttgcctgcatattcaaacagctcttctacgataagggcacaaatcgcatcgtggaacgtttgggcttctaccgatttagcagtttgatacactttctctaagtatccacctgaatcataaatcggcaaaatagagaaaaattgaccatgtgtaagcggccaatctgattccacctgagatgcataatctagtagaatctcttcgctatcaaaattcacttccaccttccactcaccggttgtccattcatggctgaactctgcttcctctgttgacatgacacacatcatctcaatatccgaatagggcccatcagtctgacgaccaagagagccataaacaccaatagccttaacatcatccccatatttatccaatattcgttccttaatttcatgaacaatcttcattctttcttctctagtcattattattggtccattcactattctcattcccttttcagataattttagatttgcttttctaaataagaatatttggagagcaccgttcttattcagctattaataactcgtcttcctaagcatccttcaatccttttaataacaattatagcatctaatcttcaacaaactggcccgtttgttgaactactctttaataaaataatttttccgttcccaattccacattgcaataatagaaaatccatcttcatcggctttttcgtcatcatctgtatgaatcaaatcgccttcttctgtgtcatcaaggtttaattttttatgtatttcttttaacaaaccaccataggagattaaccttttacggtgtaaaccttcctccaaatcagacaaacgtttcaaattcttttcttcatcatcggtcataaaatccgtatcctttacaggatattttgcagtttcgtcaattgccgattgtatatccgatttatatttatttttcggtcgaatcatttgaacttttacatttggatcatagtctaatttcattgcctttttccaaaattgaatccattgtttttgattcacgtagttttctgtattcttaaaataagttggttccacacataccaatacatgcatgtgctgattataagaattatctttattatttattgtcacttccgttgcacgcataaaaccaacaagatttttattaatttttttatattgcatcattcggcgaaatccttgagccatatctgacaaactcttatttaattcttcgccatcataaacatttttaactgttaatgtgagaaacaaccaacgaactgttggcttttgtttaataacttcagcaacaaccttttgtgactgaatgccatgtttcattgctctcctccagttgcacattggacaaagcctggatttacaaaaccacactcgatacaactttctttcgcctgtttcacgattttgtttatactctaatatttcagcacaatcttttactctttcagcctttttaaattcaagaatatgcagaagttcaaagtaatcaacattagcgattttcttttctctccatggtctcacttttccactttttgtcttgtccactaaaacccttgatttttcatctgaataaatgctactattaggacacataatattaaaagaaacccccatctatttagttatttgtttagtcacttataactttaacagatggggtttttctgtgcaaccaattttaagggttttcaatactttaaaacacatacataccaacacttcaacgcacctttcagcaactaaaataaaaatgacgttatttctatatgtatcaagataagaaagaacaagttcaaaaccatcaaaaaaagacaccttttcaggtgctttttttattttataaactcattccctgatctcgacttcgttctttttttacctctcggttatgagttagttcaaattcgttctttttaggttctaaatcgtgtttttcttggaattgtgctgttttatcctttaccttgtctacaaaccccttaaaaacgtttttaaaggcttttaagccgtctgtacgttccttaag

SEQ ID NO: 3 - DNA, 29 bp, Artificial
  Oligonucleotide Primer
ggaattcccttatgaaccaaggaatagca

SEQ ID NO: 4 - DNA, 31 bp, Artificial
  Oligonucleotide Primer
gcggccgcacccgctctttcggtaacccgct

SEQ ID NO: 5 - DNA, 31 bp, Artificial
  Oligonucleotide Primer
gcggccgcttgctaagtgaatattttcaagt

SEQ ID NO: 6 - DNA, 28 bp, Artificial
  Oligonucleotide Primer
ctgcagcgtcaattccatcacttcacga